(19) United States
(12) Patent Application Publication
(10) Pub. No.: US 2025/0213331 A1
(43) Pub. Date: Jul. 3, 2025

(54) Improvements to Dental Splints for Treatment of Snoring or Sleep Apnoea

(71) Applicant: Digital Sleep Design Pty Ltd, Burwood East, Victoria (AU)

(72) Inventors: Allan GREENHALG, Melbourne (AU); Ken LEE, Melbourne (AU); Sam TALPIS, Melbourne (AU)

(21) Appl. No.: 18/852,339

(22) PCT Filed: Mar. 31, 2023
(86) PCT No.: PCT/AU2023/050246

§ 371 Date: Sep. 27, 2024

(30) Foreign Application Priority Data
Mar. 31, 2022 (AU) 2022900837

(51) Int. Cl. A61C 7/36; A61F 5/56

(52) U.S. Cl. CPC A61C 7/36; A61F 5/566; A61F 2220/0033

(57) ABSTRACT

The present invention relates to a plug and socket set for use in a mandibular advancement splint in the treatment of snoring or sleep apnoea, the plug having a head for engagement with a mandibular abutment and the head having two prongs projecting therefrom, the socket having a substantially blind aperture configured to receive the two prongs with at least one clearance, and a crosspiece that spans the aperture for an interengagement with the two prongs, wherein the two prongs can interengage with the crosspiece to form a snap fit connection whereby a first surface and a second surface are substantially sealably mated with one another.

### PRIORITY CLAIM

The present application claims priority from Australian provisional patent application 2022900837, filed on 31 Mar. 2022. The contents of the priority application are hereby incorporated herein by reference.

### TECHNICAL FIELD

The present invention relates generally to the field of sleep apnoea, and in particular to the treatment of sleep apnoea using dental splints.

### BACKGROUND

Sleep apnoea is a sleep disorder involving pauses in breathing or periods of shallow breathing during sleep. Each pause can last from a few seconds to a few minutes. In the most common form of sleep apnoea, the pause follows loud snoring, and there may be a choking or snorting sound as breathing resumes. Since the disorder disrupts normal sleep patterns, those affected may experience sleepiness or feel tired during the day.

Obstructive sleep apnoea in particular is at least partially caused by a blockage in air flow. This may be due to excess soft tissue in the mouth, throat or airway, and one common cause is the tongue and associated connective tissue slipping into the pharynx, due to the soft, flexible and pliable nature of the tongue, aided by the mandible (the lower jaw) relaxing and subsequently retracting towards the throat. Tissues adjacent the pharynx are also more relaxed when the mandible is in a retracted position. In connection with this, the most common cause of snoring is vibration in the tissues of the upper airway as air passes over them.

There are a variety of dental appliances and treatments used to address obstructive sleep apnoea or snoring, however they all suffer from drawbacks in their various forms. One common such appliance is a dental splint that moves the mandible forward slightly (referred to as mandibular advancement), thereby slightly raising and pulling forward the tongue above the pharynx and tightening the soft tissue and muscles of the upper airway. The increased tension in the associated tissue inhibits the ability of the tongue to slip or tip backwards into the pharynx.

The typical prior art appliance consists of a pair of splints that are worn on the mandibular and maxillary dental arches, respectively. The mandibular splint is provided with wing protrusions that interface with plug and socket sets built into the maxillary splint, the adjustable plugs thereof serving to provide a mandibular advancement relative to the maxilla by abutting against the wings. The longer the plug, the further the mandible is advanced. A dentist or sleep specialist can advise on a suitable plug head size and may adjust the plug size until optimal advancement is achieved.

The plug and socket sets of the prior art are, however, cumbersome to operate as well as temperamental. They typically utilise an interference fit, which makes them difficult to join—as the skilled person will appreciate, these sets are typically very small (due to needing to fit comfortably within a person's mouth without disrupting sleep), which increases the difficulty of engaging the plug and socket through an interference-fit system. Similarly, the prior art plug is releasable by squeezing the socket, however this can be exceptionally difficult for less dexterous persons due to the socket's small size. Since the plug and socket rely upon friction to remain in an interference fit, the typical wear or simple material degradation that occurs over time may result in the plugs of the prior art becoming prone to falling out, which can create a choking hazard. The sockets are prone to fouling with bacteria due to the ingress of foodstuffs into an aperture located toward the rear of the plug, which can create a foul odour as well as increasing a friction within the plug and socket. Finally, in prior art systems the plug may also protrude through the socket which can create discomfort for the patient.

The present invention arose out of a desire to overcome or at least ameliorate some of the deficiencies of the prior art.

### DISCLOSURE OF THE INVENTION

In a first aspect, the present invention comprises a plug and socket set for use in a mandibular advancement splint, comprising a plug having a plug head of a predetermined size, a first engagement portion and a first abutment surface substantially surrounding a base thereof, and a socket having a substantially blind aperture shaped to receive the first engagement portion therewithin, a second engagement portion within the aperture, and a second abutment surface surrounding an opening of the aperture, wherein the plug head is arranged to abut against a protruding portion of the mandibular advancement splint, at least one of the first and second engagement portions are resiliently deformable to enable releasable interengagement therebetween when the first engagement portion is received within the aperture, the first and second abutment surfaces abut one another when the first engagement portion is fully received within the aperture, and the abutment between the first and second abutment surfaces is a sealing abutment.

In an embodiment, one of the first and second engagement portions comprises a protrusion, and the other of the first and second engagement portions comprises a pair of jaws adapted to releasably receive and retain the protrusion therebetween.

In an embodiment, the pair of jaws are adapted to selectively and releasably grip the protrusion, thereby retaining the protrusion therebetween, and the jaws are further able to be selectively biased apart, releasing their grip upon the protrusion that is received therebetween.

In an embodiment, the protrusion comprises a bar arranged to extend transverse to a direction of insertion into the aperture, and the pair of jaws define a space between one another having a shape substantially matching that of a cross-section of the bar.

In an embodiment, the second engagement portion is the protrusion comprising the bar, and the bar extends between two opposing inner surfaces of the aperture.

In an embodiment, the releasable interengagement comprises a snap fit connection.

In an embodiment, the aperture is a fully-blind aperture having only a single entry, and when the first and second abutment surfaces are sealingly abutted against one another, the aperture is substantially completely sealed.

In an embodiment, the plug comprises at least one further first abutment surface, and the socket comprises a matching number of further second abutment surfaces, each arranged to abut against a separate one of the at least one further first abutment surfaces when the first engagement portion is fully received within the aperture.

In an embodiment, the first abutment surface comprises a first outer perimeter, and the second abutment surface comprises a second outer perimeter shaped such that, when the first engagement portion is fully received within the aperture, at least a portion of the second outer perimeter substantially aligns with at least a portion of the first outer perimeter, further wherein an outer surface of the plug that is proximal to the first outer perimeter portion aligns with an outer surface of the socket that is proximal to the second outer perimeter portion, forming a substantially continuous outer surface of the assembled plug and socket set across the aligned portions.

In an embodiment, the second outer perimeter is a substantial mirror image of the first outer perimeter, such that, when the first engagement portion is fully received within the aperture, the first and second outer perimeters substantially completely align with one another, and substantially all outer surfaces of the plug that are proximal to the first outer perimeter align with separate particular ones of substantially all outer surfaces of the socket that are proximal to the second outer perimeter, each forming substantially continuous outer surfaces of the assembled plug and socket set across the aligned portions.

A further aspect of the invention may reside in a splint set for the treatment of snoring or sleep apnoea, comprising a maxillary splint portion shaped to fit over a user's maxillary dental arch and associated teeth, a mandibular splint portion shaped to fit over the user's mandibular dental arch and associated teeth, at least one socket extending from the maxillary splint portion, the socket having an aperture containing an engagement portion and an abutment surface surrounding an opening of the aperture, at least one protrusion extending upwardly from the mandibular splint portion, and at least one plug having a plug head of a predetermined size, a complementary engagement portion, and a complementary abutment surface, the plug being adapted to fit into the socket aperture such that the engagement portion and complementary engagement portion interengage with one another, wherein the plug head is adapted to abut or urge against the protrusion upwardly extending from the mandibular splint portion, the abutment surface and complementary abutment surface abut one another when the complementary engagement portion is fully received within the aperture, and the abutment therebetween is a sealing abutment.

In an embodiment, the at least one socket is integral with the maxillary splint portion.

In an embodiment, the set comprises a plurality of at least one plugs, each of the plurality of at least one plugs having plug heads of different predetermined sizes.

In an embodiment, the at least one plug and the at least one socket are at least one plug and socket of the plug and socket set of an embodiment of the first aspect of the invention.

In an embodiment, the at least one socket is a pair of sockets arranged on opposing sides of the maxillary splint portion, the at least one protrusion is a pair of protrusions, each one extending upwardly from opposing sides of the mandibular splint portions, and the at least one plug is a pair of plugs having plug heads of the same predetermined size.

A further aspect of the invention may reside in a method of producing a splint set for the treatment of snoring or sleep apnoea in a user, comprising the steps of:
i. forming, using a mould of the user's maxillary dental arch and associated teeth, a maxillary splint portion having at least one socket extending therefrom, the socket having an aperture containing an engagement portion and an abutment surface surrounding an opening of the aperture;
ii. forming, using a mould of the user's mandibular dental arch and associated teeth, a mandibular splint portion having at least one protrusion extending upwardly therefrom; and
iii. providing at least one plug, the plug having a plug head of a predetermined size, a complementary engagement portion, and a complementary abutment surface;
   wherein the plug is adapted to fit into the socket aperture such that the engagement portion and complementary engagement portion interengage with one another, the plug head is adapted to urge against the protrusion upwardly extending from the mandibular splint portion, the abutment surface and complementary abutment surface abut one another when the complementary engagement portion is fully received within the aperture, and the abutment therebetween is a sealing abutment.

In an embodiment, the step of forming the maxillary splint portion having at least one socket extending therefrom comprises the sub-steps of:
a. Casting or otherwise forming a mould of the user's maxillary arch and associated teeth;
b. forming the maxillary splint portion using the mould;
c. separately forming the at least one socket; and
d. welding, gluing or otherwise affixing the at least one socket to the maxillary splint portion.

In an embodiment, the at least one socket is integral with the maxillary splint portion, and the step of forming the maxillary splint portion having at least one socket extending therefrom comprises the sub-steps of:
a. Casting or otherwise forming a mould of the user's maxillary arch and associated teeth;
b. Adapting the mould to further comprise at least one void having a substantially identical shape to that of the at least one socket; and
c. forming the maxillary splint portion and at least one socket.

In an embodiment, the at least one socket is a pair of sockets arranged on opposing sides of the maxillary splint portion, the at least one protrusion is a pair of protrusions, each one extending upwardly from opposing sides of the mandibular splint portions, and the at least one plug is a pair of plugs having plug heads of the same predetermined size.

In an embodiment, the splint set is a splint set of an embodiment of the second aspect of the invention.

In an embodiment, the at least one plug and the at least one socket are at least one plug and socket of the plug and socket set of an embodiment of the first aspect of the invention.

A further aspect of the invention may reside in a method of treatment of snoring or sleep apnoea in a user, comprising the steps of:
i. providing, to the user, the splint set having a maxillary splint portion shaped to fit over the user's maxillary dental arch and associated teeth, a mandibular splint portion shaped to fit over the user's mandibular dental arch and associated teeth, at least one socket extending from the maxillary splint portion, the socket having an aperture containing an engagement portion and an abutment surface surrounding an opening of the aperture, at least one protrusion extending upwardly from the mandibular splint portion, and at least one plug having a plug head of a predetermined size, a complementary engagement portion, and a complementary abutment surface;
ii. fitting the plug into the socket, such that the engagement portion and complementary engagement portion interengage with one another and the abutment surface and complementary abutment surface form a sealing abutment;
iii. fitting the maxillary splint portion to the user's maxillary dental arch; and
iv. fitting the mandibular splint portion to the user's mandibular dental arch with the upwardly-extending protrusions aligned with the plug heads;
   such that, in use, the user's mandible is inhibited from backwards movement by the upwardly-extending protrusions abutting or urging against the plug heads.

In an embodiment, the splint set is produced through an embodiment of a method of the third aspect of the invention.

In an embodiment, the splint set is a splint set of an embodiment of the second aspect of the invention.

In an embodiment, the at least one plug and the at least one socket are at least one plug and socket of the plug and socket set of an embodiment of the first aspect of the invention.

Further aspects and/or embodiments of the invention may be disclosed herein or may otherwise become apparent to the person skilled in the art through the disclosure herein. These and other embodiments are considered to fall within the scope and object of the invention.

### DESCRIPTION OF FIGURES

Embodiments of the present invention will now be described in relation to figures, wherein:

FIG. 1 is a depiction of an embodiment of a plug and socket set in relation to a mandibular advancement splint;

FIG. 2 depicts an embodiment of the plug and the socket of the plug and socket set;

FIG. 3 depicts an internal view of the plug and socket of FIG. 2 in engagement with one another;

FIG. 4 depicts an embodiment of a mandibular advancement splint of the present invention;

FIG. 5 depicts an embodiment of the socket of the plug and socket set, showing the drainage aperture therein;

FIG. 6 depicts a range of plugs having different head sizes; and

FIG. 7 depicts an assembled embodiment of a mandibular advancement splint.

### DETAILED DESCRIPTION OF PREFERRED EMBODIMENTS

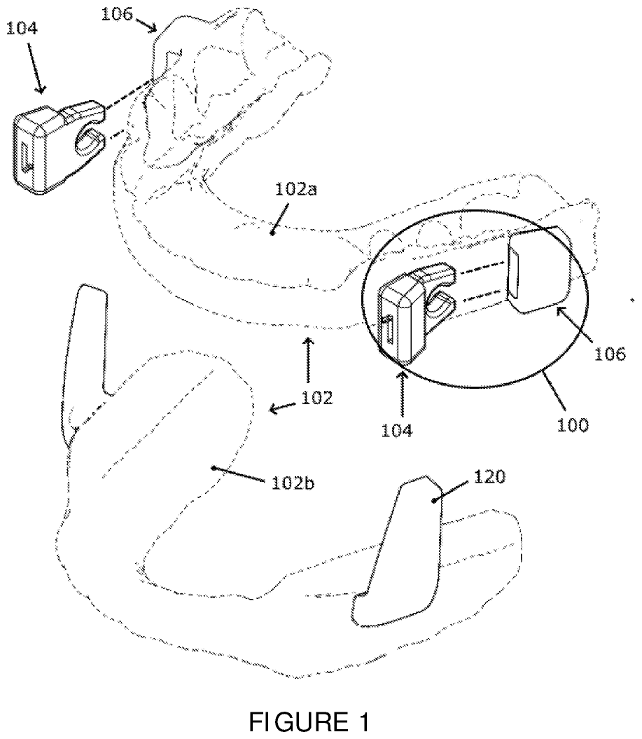

In an embodiment of the invention and with reference to FIGS. 1 & 2, there is provided a plug and socket set 100 for use in a mandibular advancement splint 102. The set 100 comprises a plug 104 and a complementary socket 106. In an embodiment, the plug 104 comprises a plug head 108 of a predetermined size, a first engagement portion 110, and a first abutment surface 112 substantially surrounding a base of the first engagement portion. In an embodiment, the socket 106 comprises a substantially blind aperture 114 that is shaped to receive the first engagement portion 110 therewithin, a second engagement portion 116 within the aperture, and a second abutment surface 118 surrounding an opening of the aperture. In an embodiment, at least one of the first and second engagement portions 110, 116 are resiliently deformable to enable releasable interengagement therebetween when the first engagement portion 110 is received within the aperture 114. In an embodiment, the plug head 108 is arranged to abut against a protruding portion 120 of the mandibular advancement splint 102. In an embodiment, the first and second abutment surfaces 112, 118 abut one another when the first engagement portion 110 is fully received within the aperture 114.

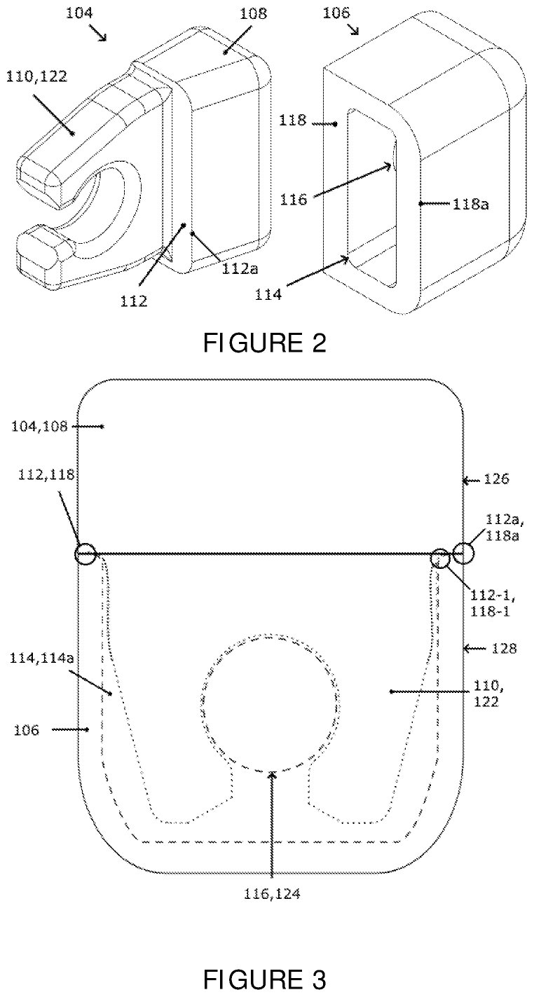

In an embodiment and with reference to FIGS. 2 & 3, one of the first and second engagement portions 110, 116 may comprise a protrusion 124, and the other may comprise a pair of jaws 122 adapted to releasably receive and retain the protrusion therebetween. FIGS. 1-3 depict the first engagement portion 110 as comprising a pair of jaws 122, however the person skilled in the art will appreciate that this is exemplary only and that the second engagement portion 116 can be adapted to comprise a pair of jaws without departing from the scope of the invention.

In a further embodiment, the pair of jaws 122 are adapted to selectively and releasably grip the protrusion 124, thereby retaining the protrusion therebetween. This is particularly depicted in FIG. 3, which illustrates the plug 104 received by the socket 106 with the internal space within the aperture 114 shown. The dashed lines depict the internal surface of the aperture 114 and the second engagement portion 116, shown herein as being comprising the protrusion 124. The dotted lines depict the first engagement portion 110 of the plug 104, shown herein as comprising a pair of jaws 122 that have releasably received the protrusion 124 therebetween and retain the protrusion in engagement therewith. In an embodiment, this may be achieved by the jaws 122 being at least partially comprised of a resiliently-flexible material shaped such that, at rest, the jaws are in a ‘closed’ state surrounding and retaining the protrusion 124, but an application of force to the plug 104 and/or socket 106 in opposing directions will result in the jaws biasing open and allowing the protrusion to be withdrawn therefrom. This may enable embodiments of the present invention to be more easily adjusted by users compared to the prior art, particularly given the typically small size of the plug and socket set 100.

In some embodiments, the protrusion 124 may comprise a ball, orb or other bulbous or otherwise shaped protrusion that is able to be gripped between a pair of jaws without slipping back out. In alternate embodiments, the protrusion may comprise a bar that is substantially non-parallel to the direction of insertion of the plug 104 into engagement with the socket 106, with the pair of jaws 122 defining a space between one another having a shape substantially matching that of a cross-section of the bar. In one further embodiment wherein the second engagement portion 116 is the protrusion 124 comprising the bar and as depicted in FIG. 3, the bar may extend between two opposing inner surfaces of the aperture.

In an embodiment, the releasable interengagement between the first and second engagement portions 110, 116 may comprise a snap-fit connection. Such a snap-fit connection may be able to be disengaged using a tool such as a screwdriver as a wedge. One example embodiment may be able to be levered apart by using the tip of a typical flat-head screwdriver: by inserting the tip of the screwdriver between the plug 104 and the socket 102 and twisting it, the plug and socket can be levered apart. The person skilled in the art will appreciate that-given the miniscule size of the plug and socket set 100—this may be an easier disengagement method for a user compared to needing to apply pressure on opposing sides of the plug or socket while pulling on the other. This may be of particular benefit to users who have lost some dexterity or otherwise are less mobile.

In an embodiment and as depicted in FIG. 3, the abutment between the first and second abutment surfaces 112, 118 may be a sealing abutment.

However, it is well known in the art that an aperture 114 may ordinarily provide an area for particulate and/or bacteria to accumulate-plug-and-socket connections are typically avoided in dental devices due to the socket providing a cavity for accumulation of food particulate and/or bacteria.

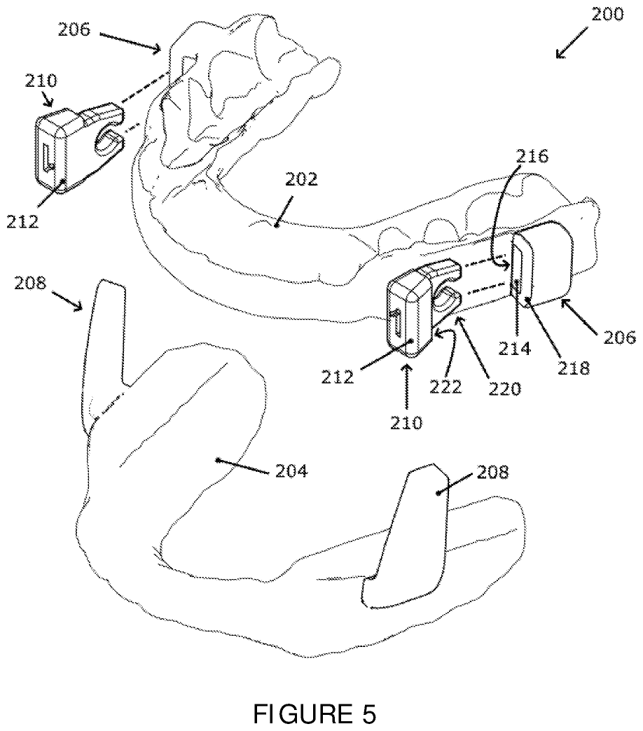

In order to reduce or mitigate the accumulation of food particulate, but particularly to allow for saliva to drain from the socket (and most particularly aperture 114 of the socket), in an embodiment of the present invention that is best shown in FIG. 5, the socket 106 may include a drainage aperture therein, which allows for passageway of dental fluids to pass therethrough, and so drain from the socket.

In some embodiments, it may be desirable to shape the aperture 114 to have an internal shape larger than that of the first engagement portion 110 that extends thereinto upon engagement—it is considered that the increased size of the aperture 114 may provide a clearance space 114a for the movement of the first engagement portion 110, such that friction and other resistive elements do not impede the engagement of the first and second engagement portions 110, 116. It is further considered that providing such a clearance space 114a, which would ordinarily provide refuge for particulate and/or bacteria, may be enabled by provision of a sealing abutment between the first and second abutment surfaces 112, 118.

In an embodiment, the shapes and topology of the first and second abutment surfaces 112, 118 are substantially complementary. This may comprise—as depicted in FIG. 3—first and second abutment surfaces 112, 118 being ‘true’ abutment surfaces that are each perpendicular to the direction of insertion of the plug 104 into the socket 106. In alternate embodiments, the substantially complementary first and second abutment surfaces 112, 118 may comprise curved surfaces, angled surfaces or any other arrangement of complementary surfaces capable of forming a sealing abutment.

In a further embodiment, the plug may comprise at least one further first abutment surface 112-1, and the socket may comprise a matching number of further second abutment surfaces 118-1, with each being arranged to abut against a separate one of the at least one further first abutment surfaces when the first engagement portion is fully received within the aperture. With reference to FIG. 3, one example of a further first abutment surface 112-1 may comprise a surface adjacent to the first abutment surface 112, with the matching further second abutment surface 118-1 being similarly arranged relative to the second abutment surface 118.

In an embodiment, the aperture 114 may be fully blind whereby the aperture opening is the only means for a fluid, particulate or other material to ingress into or egress from the aperture 114. This, combined with the sealing abutment between the first and second abutment surfaces 112, 118 and further first and second abutment surfaces 112-1, 118-1 (if present) may substantially ameliorate or alleviate the risk of particulate or bacteria accumulation within the aperture 114. Advantageously, this may enable an embodiment comprising a clearance space 114a within the aperture 114 to have air trapped and sealed therewithin and surrounding the engaged first and second engagement portions 110, 116, which may further reduce the possibility of bacterial fouling therein by providing an environment hostile to typical oral bacteria.

In an embodiment and with return reference to FIG. 2 and further reference to FIG. 3, the first abutment surface 112 may comprise a first outer perimeter 112a, and the second abutment surface 118 may comprise a second outer perimeter 118a that is shaped such that, when the first engagement portion is fully received within the aperture, at least a portion of the second outer perimeter substantially aligns with at least a portion of the first outer perimeter. In such an embodiment, an outer surface 126 of the plug 104 that is proximal to the first outer perimeter portion 112a aligns with an outer surface 128 of the socket 106 that is proximal to the second outer perimeter portion 118a, forming a substantially continuous outer surface of the assembled plug and socket set across the aligned portions. Such an embodiment may be advantageous as discontinuous surfaces are known to form ridges and/or valleys wherein bacteria and/or particulate are able to accumulate. By providing substantially-continuous outer surfaces, the accumulation of bacteria and/or particulate may be substantially ameliorated.

In a further embodiment, the second outer perimeter 118a may be a substantial mirror image of the first outer perimeter 112a, such that, when the first engagement portion is fully received within the aperture, the first and second outer perimeters substantially completely align with one another. In such an embodiment, substantially all outer surfaces 126 of the plug 104 that are proximal to the first outer perimeter 112a may align with separate particular ones of substantially all outer surfaces 128 of the socket 106 that are proximal to the second outer perimeter 118a, each forming substantially continuous outer surfaces of the assembled plug and socket set 100 across the aligned portions.

With return reference to FIG. 1, the plug head 108 is adapted to abut and/or urge against the protrusions 120 that upwardly extend from the mandibular splint portion 102b. In use, the user's mandible is thus urged forwards, often referred to as mandibular advancement. In some embodiments, the plug head 108 may be provided in a predetermined size, said size being selected for a desired level of mandibular advancement. In such an embodiment, the ability to remove the plug 104 from within the socket 106 plug and socket set 100 enables the extent of mandibular advancement to be suitably selected from the outset or simply adjusted by the replacement of one plug 104 with another of a different size.

In an embodiment, an outer surface of the plug 104 or socket 106 may be finished in order to reduce the presence of roughened surfaces, pitting or other imperfections. As the person skilled in the art will appreciate, roughened surfaces—like discontinuous surfaces—can provide harbour for bacteria or particulate. Additionally, some methods of production such as 3D printing or injection moulding may be prone to pitting, which provide similar refuge for particulate matter and/or bacteria. It is therefore considered to be beneficial, in at least some embodiments wherein the material of the plug and socket set 100 is receptive to surface finishing, to finish at least one outer surface thereof. Finishing may include filing, grinding, sanding, polishing, or any combination thereof. The finish may ‘smooth out’ rough areas, thereby inhibiting the ability for bacteria or particulate such as food particulate to become entrained thereagainst. Finishing may also alleviate the effects of pitting or other manufacturing defects that may be present in an outer surface of the plug and socket set.

In an embodiment, the plug 104 or socket 106 may be finished through vapour polishing, wherein a chemical vapour is blown over the outer surface, which induces melting and plastic flow in the outer layers thereof to form a vapour-polished outer surface. This may enable surface micro-features (scratches, roughness and other imperfections only visible at the microscopic level) to smooth out as a result of the molten plastic flow, and may be beneficial in certain embodiments wherein the manufacturing process may induce these. In an embodiment, the chemical vapour may be a vaporised form of a plastic solvent. In an embodiment, the vapour polished outer surface may be substantially or essentially resistant to entrainment or deposition of particulate matter and/or bacteria thereupon. In an embodiment, vapour polishing may be combined with one or more other types of surface finish to address one or more other surface defects that may not be readily addressable through the vapour polishing process.

A second aspect of the invention may lie in a splint set 200 for the treatment of snoring or sleep apnoea. In an embodiment, this may comprise a maxillary splint portion 202 shaped to fit over a user's maxillary dental arch and associated teeth, a mandibular splint portion 204 shaped to fit over the user's mandibular dental arch and associated teeth, at least one socket 206 extending from the maxillary splint portion, at least one protrusion 208 extending upwardly from the mandibular splint portion, and at least one plug 210 having a plug head 212 of a predetermined size. In an embodiment, the socket 206 may comprise an aperture 214 containing an engagement portion 216 (inside) and an abutment surface 218 surrounding an opening of the aperture, and the plug 210 may comprise a complementary engagement portion 220 and a complementary abutment surface 222. In such an embodiment, the plug may be adapted to fit into the socket aperture 214 such that the engagement portion 216 and complementary engagement portion 220 interengage with one another. The abutment surface 218 and complementary abutment surface 222 may abut one another when the complementary engagement portion 220 is fully received within the aperture 214, forming a sealing abutment. The plug head 212 may be adapted to abut or urge against the protrusion 208 that extends upwardly from the mandibular splint portion 204.

In an embodiment, the at least one socket 206 may be integral with the maxillary splint portion 202. In such an embodiment, the maxillary splint portion 202 may be formed by using a mould of the intended user's maxillary dental arch and associated teeth, and prior to casting the maxillary splint portion 202, the mould may be adapted to provide the socket 206. This may promote structural rigidity and strength of the splint set and may prevent or ameliorate the risk of the socket 206 and/or plug 210 becoming detached from the rest of the splint set 200.

In an embodiment and with reference to FIG. 5, the splint set 200 may comprise a plurality of at least one plugs 210, each of the plurality of at least one plugs 210 having a plug head 212 of different predetermined sizes. FIG. 5 depicts a group of four plugs 210-1-210-4, each having different predetermined plug head sizes 212-1-212-4. As the skilled person will appreciate, the other features of the plugs 210-1-210-4 remain the same such that any one plug may be selected to be fitted into the same socket 206.

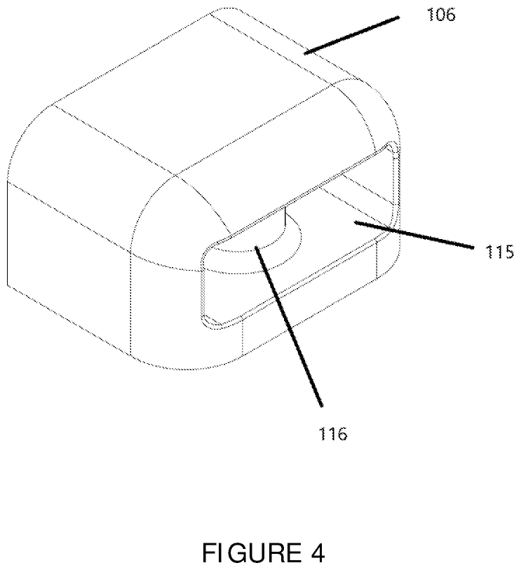

In an embodiment and with return reference to FIG. 4, the splint set 200 may comprise a pair of sockets 206 arranged on opposing sides of the maxillary splint portion 202, a pair of protrusions 208, each one upwardly extending from opposing sides of the mandibular splint portion 204, and a pair of plugs 210 having plug heads 212 of the same predetermined size. In a further embodiment, the set may comprise a plurality of pairs of plugs, each separate one of the plurality of pairs of plugs having a different associated plug head size. The skilled person will appreciate that both plugs in the same pair of plugs have the same plug head size.

In an embodiment, the at least one plug 210 and the at least one socket 206 may be a plug 104 and socket 106 of an embodiment of the first aspect of the invention.

A further aspect of the invention may lie in a method of producing a splint set for the treatment of snoring or sleep apnoea in a user. In an embodiment, the method may comprise the steps of:
i. forming, using a mould of the user's maxillary dental arch and associated teeth, a maxillary splint portion having at least one socket extending therefrom, the socket having an aperture containing an engagement portion and an abutment surface surrounding an opening of the aperture;
ii. forming, using a mould of the user's mandibular dental arch and associated teeth, a mandibular splint portion having at least one protrusion extending upwardly therefrom; and
iii. providing at least one plug, the plug having a plug head of a predetermined size, a complementary engagement portion, and a complementary abutment surface.

In an embodiment, the plug is adapted to fit into the socket aperture such that the engagement portion and complementary engagement portion interengage with one another. In an embodiment, the plug head is adapted to urge against the protrusion that upwardly extends from the mandibular splint portion. In an embodiment, the abutment surface and complementary abutment surface abut one another when the complementary engagement portion is fully received within the aperture and form a sealing abutment.

In an embodiment, the step of forming the maxillary splint portion having at least one socket extending therefrom may comprise the sub-steps of:
a. Casting or otherwise forming a mould of the user's maxillary arch and associated teeth;
b. forming the maxillary splint portion using the mould;
c. separately forming the at least one socket; and
d. welding, gluing or otherwise affixing the at least one socket to the maxillary splint portion.

In an alternate embodiment, the at least one socket is integral with the maxillary splint portion. In such an embodiment, the step of forming the maxillary splint portion having at least one socket extending therefrom may comprise the sub-steps of:
a. Casting or otherwise forming a mould of the user's maxillary arch and associated teeth;
b. Adapting the mould to further comprise at least one void having a substantially identical shape to that of the at least one socket; and
c. forming the maxillary splint portion and at least one socket.

In an embodiment, the at least one socket is a pair of sockets arranged on opposing sides of the maxillary splint portion, the at least one protrusion is a pair of protrusions, each one extending upwardly from opposing sides of the mandibular splint portions, and the at least one plug is a pair of plugs having plug heads of the same predetermined size.

In an embodiment, the method may further comprise the step of finishing an outer surface of at least one of the maxillary splint portion, the mandibular splint portion, the at least one socket, the at least one plug, or the at least one protrusion, wherein finishing includes filing, grinding, sanding, polishing, or any combination thereof. This may provide a finished outer surface that is resistant to catching bacteria or particulate matter thereupon. In a further embodiment, the step of finishing the outer surface may comprise at least vapour polishing the outer surface. This may be in combination with one or more additional finishing processes.

In an embodiment, the splint set produced by the method may be a splint set 200 of an embodiment of the second aspect of the invention.

In an embodiment, the at least one plug and the at least one socket may be a plug 104 and socket 106 of an embodiment of the first aspect of the invention.

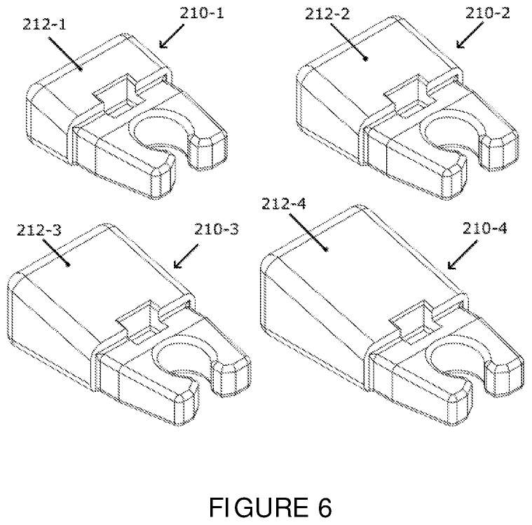

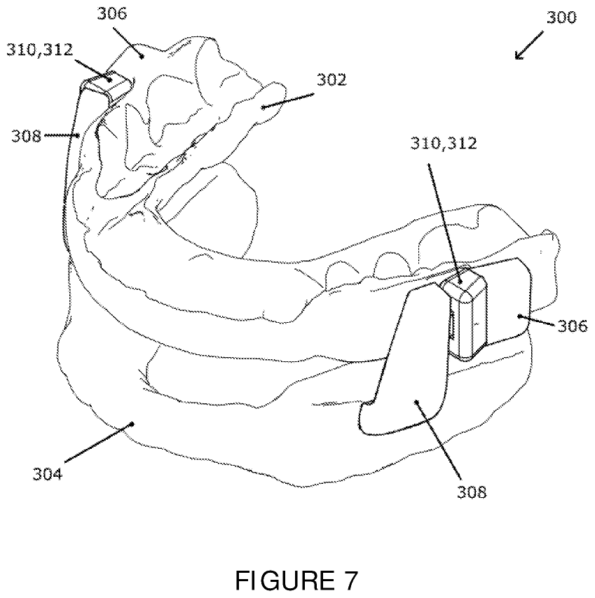

With reference to FIG. 6, a further aspect of the invention may lie in a method of treatment of snoring or sleep apnoea in a user. In an embodiment, the method may comprise the steps of:
i. providing, to the user, a splint set 300 having a maxillary splint portion 302 shaped to fit over the user's maxillary dental arch and associated teeth, a mandibular splint portion 304 shaped to fit over the user's mandibular dental arch and associated teeth, at least one socket 306 extending from the maxillary splint portion, the socket having an aperture containing an engagement portion and an abutment surface surrounding an opening of the aperture, at least one protrusion 308 extending upwardly from the mandibular splint portion, and at least one plug 310 having a plug head 312 of a predetermined size, a complementary engagement portion, and a complementary abutment surface;
ii. fitting the plug 310 into the socket 306, such that the engagement portion and complementary engagement portion interengage with one another and the abutment surface and complementary abutment surface form a sealing abutment;
iii. fitting the maxillary splint portion 302 to the user's maxillary dental arch; and
iv. fitting the mandibular splint portion 304 to the user's mandibular dental arch with the upwardly-extending protrusions 308 aligned with the plug heads 312.

In use and with further reference to FIG. 6, the user's mandible (not depicted) is inhibited from backwards movement by the upwardly-extending protrusions 308 abutting or urging against the plug heads 312. In further embodiments, the user's mandible may be held slightly forwards of an otherwise natural resting position (mandibular advancement) to prevent or inhibit occlusion of the user's airway during sleep.

In an embodiment, the splint set 300 may be produced by a method of an embodiment of the third aspect of the invention.

In an embodiment, the splint set 300 may be a splint set 200 of an embodiment of the second aspect of the invention.

In an embodiment, the at least one plug 310 and the at least one socket 306 may be a plug 104 and socket 106 of an embodiment of the first aspect of the invention.

While the invention has been described with reference to preferred embodiments above, it will be appreciated by those skilled in the art that it is not limited to those embodiments, but may be embodied in many other forms, variations and modifications other than those specifically described. The invention includes all such variation and modifications. The invention also includes all of the steps, features, components and/or devices referred to or indicated in the specification, individually or collectively and any and all combinations or any two or more of the steps or features.

In this specification, unless the context clearly indicates otherwise, the word “comprising” is not intended to have the exclusive meaning of the word such as “consisting only of”, but rather has the non-exclusive meaning, in the sense of “including at least”. The same applies, with corresponding grammatical changes, to other forms of the word such as “comprise”, etc.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Any promises made in the present document should be understood to relate to some embodiments of the invention, and are not intended to be promises made about the invention in all embodiments. Where there are promises that are deemed to apply to all embodiments of the invention, the applicant/patentee reserves the right to later delete them from the description and they do not rely on these promises for the acceptance or subsequent grant of a patent in any country.

1. A plug and socket set for use in a mandibular advancement splint, comprising:
a plug having a plug head of a predetermined size, a first engagement portion and a first abutment surface substantially surrounding a base thereof; and
a socket having:
a substantially blind aperture shaped to receive the first engagement portion therewithin;
a second engagement portion within the aperture; and
a second abutment surface surrounding an opening of the aperture;
wherein the plug head is arranged to abut against a protruding portion of the mandibular advancement splint;
at least one of the first and second engagement portions are resiliently deformable to enable releasable interengagement therebetween when the first engagement portion is received within the aperture;
the first and second abutment surfaces abut one another when the first engagement portion is fully received within the aperture; and
the abutment between the first and second abutment surfaces is a sealing abutment.

2. The plug and socket set of claim 1, wherein:
one of the first and second engagement portions comprises a protrusion; and
the other of the first and second engagement portions comprises a pair of jaws adapted to releasably receive and retain the protrusion therebetween.

3. The plug and socket set of claim 2, wherein the pair of jaws are adapted to selectively and releasably grip the protrusion, thereby retaining the protrusion therebetween; and
the jaws are further able to be selectively biased apart, releasing their grip upon the protrusion that is received therebetween.

4. The plug and socket set of either claim 2 or 3, wherein the protrusion comprises a bar arranged to extend transverse to a direction of insertion into the aperture; and
the pair of jaws define a space between one another having a shape substantially matching that of a cross-section of the bar.

5. The plug and socket set of claim 4, wherein the second engagement portion is the protrusion comprising the bar; and
the bar extends between two opposing inner surfaces of the aperture.

6. The plug and socket set of any one of the above claims, wherein the releasable interengagement comprises a snap fit connection.

7. The plug and socket set of any one of the above claims, wherein the aperture is a fully-blind aperture having only a single entry; and
when the first and second abutment surfaces are sealingly abutted against one another, the aperture is substantially completely sealed against fluid and particulate ingress thereinto or egress therefrom.

8. The plug and socket set of any one of the above claims, wherein the plug comprises at least one further first abutment surface; and
the socket comprises a matching number of further second abutment surfaces, each arranged to abut against a separate one of the at least one further first abutment surfaces when the first engagement portion is fully received within the aperture.

9. The plug and socket set of any one of the above claims, wherein the first abutment surface comprises a first outer perimeter; and
the second abutment surface comprises a second outer perimeter shaped such that, when the first engagement portion is fully received within the aperture, at least a portion of the second outer perimeter substantially aligns with at least a portion of the first outer perimeter;
further wherein an outer surface of the plug that is proximal to the first outer perimeter portion aligns with an outer surface of the socket that is proximal to the second outer perimeter portion, forming a substantially continuous outer surface of the assembled plug and socket set across the aligned portions.

10. The plug and socket set of claim 9, wherein:
the second outer perimeter is a substantial mirror image of the first outer perimeter, such that, when the first engagement portion is fully received within the aperture, the first and second outer perimeters substantially completely align with one another; and
substantially all outer surfaces of the plug that are proximal to the first outer perimeter align with separate particular ones of substantially all outer surfaces of the socket that are proximal to the second outer perimeter, each forming substantially continuous outer surfaces of the assembled plug and socket set across the aligned portions.

11. The plug and socket set of any one of the above claims, wherein at least one of the plug and the socket comprise a finished outer surface; and
the finished outer surface is finished through one or more of filing, grinding, sanding or polishing.

12. The plug and socket set of claim 11, wherein the finished outer surface is a vapor polished outer surface.

13. A splint set for the treatment of snoring or sleep apnoea, comprising:
a maxillary splint portion shaped to fit over a user's maxillary dental arch and associated teeth;
a mandibular splint portion shaped to fit over the user's mandibular dental arch and associated teeth;
at least one socket extending from the maxillary splint portion, the socket having an aperture containing an engagement portion and an abutment surface surrounding an opening of the aperture;
at least one protrusion extending upwardly from the mandibular splint portion; and
at least one plug having a plug head of a predetermined size, a complementary engagement portion, and a complementary abutment surface, the plug being adapted to fit into the socket aperture such that the engagement portion and complementary engagement portion interengage with one another;
wherein the plug head is adapted to abut or urge against the protrusion upwardly extending from the mandibular splint portion;
the abutment surface and complementary abutment surface abut one another when the complementary engagement portion is fully received within the aperture; and
the abutment therebetween is a sealing abutment.

14. The splint set of claim 13, wherein the at least one socket is integral with the maxillary splint portion.

15. The splint set of either claim 13 or 14, wherein the set comprises a plurality of at least one plugs, each of the plurality of at least one plugs having plug heads of different predetermined sizes.

16. The splint set of any one of claims 13-15, wherein the at least one plug and the at least one socket are at least one plug and socket of the plug and socket set of any one of claims 1-12.

17. The splint set of any one of claims 11-16, wherein:
the at least one socket is a pair of sockets arranged on opposing sides of the maxillary splint portion;
the at least one protrusion is a pair of protrusions, each one extending upwardly from opposing sides of the mandibular splint portions; and
the at least one plug is a pair of plugs having plug heads of the same predetermined size.

18. A method of producing a splint set for the treatment of snoring or sleep apnoea in a user, comprising the steps of:
(I) forming, using a mould of the user's maxillary dental arch and associated teeth, a maxillary splint portion having at least one socket extending therefrom, the socket having an aperture containing an engagement portion and an abutment surface surrounding an opening of the aperture;
(II) forming, using a mould of the user's mandibular dental arch and associated teeth, a mandibular splint portion having at least one protrusion extending upwardly therefrom; and
(III) providing at least one plug, the plug having a plug head of a predetermined size, a complementary engagement portion, and a complementary abutment surface;
wherein the plug is adapted to fit into the socket aperture such that the engagement portion and complementary engagement portion interengage with one another;
the plug head is adapted to urge against the protrusion upwardly extending from the mandibular splint portion;
the abutment surface and complementary abutment surface abut one another when the complementary engagement portion is fully received within the aperture; and
the abutment therebetween is a sealing abutment.

19. The method of claim 18, wherein the step of forming the maxillary splint portion having at least one socket extending therefrom comprises the sub-steps of:
a. Casting or otherwise forming a mould of the user's maxillary arch and associated teeth;
b. forming the maxillary splint portion using the mould;
C. separately forming the at least one socket; and
d. welding, gluing or otherwise affixing the at least one socket to the maxillary splint portion.

20. The method of claim 18, wherein the at least one socket is integral with the maxillary splint portion; and
the step of forming the maxillary splint portion having at least one socket extending therefrom comprises the sub-steps of:
a. Casting or otherwise forming a mould of the user's maxillary arch and associated teeth;
b. Adapting the mould to further comprise at least one void having a substantially identical shape to that of the at least one socket; and
c. forming the maxillary splint portion and at least one socket.

21. The method of any one of claims 18-20, wherein:
the at least one socket is a pair of sockets arranged on opposing sides of the maxillary splint portion;
the at least one protrusion is a pair of protrusions, each one extending upwardly from opposing sides of the mandibular splint portions; and
the at least one plug is a pair of plugs having plug heads of the same predetermined size.

22. The method of any one of claims 18-21, further comprising the step of finishing an outer surface of at least one of the maxillary splint portion, the mandibular splint portion, the at least one socket, the at least one plug, or the at least one protrusion;
wherein finishing includes filing, grinding, sanding, polishing, or any combination thereof.

23. The method of claim 22 wherein the step of finishing the outer surface comprises at least vapour polishing the outer surface.

24. The method of any one of claims 18-23, wherein the splint set is a splint set of any one of claims 13-17.

25. The method of any one of claims 18-24, wherein the at least one plug and the at least one socket are at least one plug and socket of the plug and socket set of any one of claims 1-12.

26. A method of treatment of snoring or sleep apnoea in a user, comprising the steps of:
(I) providing, to the user, the splint set having a maxillary splint portion shaped to fit over the user's maxillary dental arch and associated teeth, a mandibular splint portion shaped to fit over the user's mandibular dental arch and associated teeth, at least one socket extending from the maxillary splint portion, the socket having an aperture containing an engagement portion and an abutment surface surrounding an opening of the aperture, at least one protrusion extending upwardly from the mandibular splint portion, and at least one plug having a plug head of a predetermined size, a complementary engagement portion, and a complementary abutment surface;
(II) fitting the plug into the socket, such that the engagement portion and complementary engagement portion interengage with one another and the abutment surface and complementary abutment surface form a sealing abutment;
(III) fitting the maxillary splint portion to the user's maxillary dental arch; and
(IV) fitting the mandibular splint portion to the user's mandibular dental arch with the upwardly-extending protrusions aligned with the plug heads;
such that, in use, the user's mandible is inhibited from backwards movement by the upwardly-extending protrusions abutting or urging against the plug heads.

27. The method of claim 26, wherein the splint set is produced through a method of any one of claims 18-25.

28. The method of claim 26 or 27, wherein the splint set is a splint set of any one of claims 13-17.

29. The method of any one of claims 26-28, wherein the at least one plug and the at least one socket are at least one plug and socket of the plug and socket set of any one of claims 1-12.
